SEQUENCE LISTING (19 sequences)

SEQ ID NO: 1 - protein, 121 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 2 - protein, 160 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 3 - protein, 198 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 4 - protein, 237 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 5 - protein, 275 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 6 - protein, 314 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 7 - protein, 352 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 8 - protein, 391 aa, Artificial Sequence
  SYNTHETIC SEQUENCE
AVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 9 - protein, 122 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 10 - protein, 160 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 11 - protein, 199 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 12 - protein, 237 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 13 - protein, 276 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 14 - protein, 314 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 15 - protein, 353 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSK

SEQ ID NO: 16 - protein, 391 aa, Artificial Sequence
  SYNTHETIC SEQUENCE
AVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSFNMQCQRRFYEALHDPNLTEEQRNAKIQSIRDDCAVAQSFNMQQQRRFYEALHDPNLTEEQRNAKIQSIRDDAVAQSK

SEQ ID NO: 17 - protein, 33 aa, Artificial Sequence
  SYNTHETIC SEQUENCE
FNMQQQRRFYEALHDPNLTEEQRNAKIQSIRDD

SEQ ID NO: 18 - protein, 34 aa, Artificial Sequence
  SYNTHETIC SEQUENCE
FNMQCQRRFYEALHDPNLTEEQRNAKIQSIRDDC

SEQ ID NO: 19 - protein, 5 aa, Artificial Sequence
  SYNTHETIC SEQUENCE
AVAQS

(19) United States
(12) Patent Application Publication
(10) Pub. No.: US 2023/0114372 A1
(43) Pub. Date: Apr. 13, 2023

(54) POLYPEPTIDE AFFINITY LIGANDS AND METHODS OF USING

(71) Applicant: AVANTOR PERFORMANCE MATERIALS, LLC, Radnor, PA (US)

(72) Inventors: Quanxuan ZHANG, Green Brook, NJ (US); Joshua SUMOSKI, Somerville, NJ (US); Jungmin OH, Somerville, NJ (US); Hong LI, Plainsboro, NJ (US); Jonathan FURA, Macungie, PA (US); Nandu DEORKAR, Cedar Knolls, NJ (US)

(73) Assignee: AVANTOR PERFORMANCE MATERIALS, LLC, Radnor, PA (US)

(21) Appl. No.: 17/911,322

(22) PCT Filed: Feb. 5, 2021
(86) PCT No.: PCT/US21/16890

§ 371 Date: Sep. 13, 2022

Related U.S. Application Data
(60) Provisional application No. 62/971,509, filed on Feb. 7, 2020.
(60) Provisional application No. 63/064,213, filed on Aug. 11, 2020.

(51) Int. Cl. C07K 1/22; C07K 17/06; C07K 16/00; C07K 14/705; B01D 15/20; B01D 15/36; B01D 15/32; B01D 15/38; B01J 20/32

(52) U.S. Cl. CPC C07K 1/22; C07K 17/06; C07K 16/00; C07K 14/70503; B01D 15/20; B01D 15/361; B01D 15/327; B01D 15/3809; B01J 20/3274

(57) ABSTRACT

The present invention relates to a novel polypeptide affinity ligand coupled to solid supports and affinity purification of IgG antibodies. The invention is comprised of (1) the design, generation, and purification of polypeptide ligands, (2) coupling of a polypeptide affinity ligand to a solid support matrix, (3) purification of IgG (polyclonal and monoclonal antibodies), and (4) cleaning and reuse of polypeptide supported solid matrix.

### CROSS-REFERENCE TO RELATED APPLICATIONS

This application claims the benefit of U.S. Provisional Application No. 63/064,213, filed Aug. 11, 2020, and U.S. Provisional Application No. 62/971,509, filed Feb. 7, 2020, both of which are hereby incorporated by reference in their entireties.

### FIELD OF THE INVENTION

The invention relates to polypeptide affinity ligands that have high affinity for proteins, particularly for immunoglobulin proteins, and to solid supports to which such ligands are attached, and to methods for purification of proteins, particularly immunoglobulin proteins (e.g., IgG (polyclonal or monoclonal) antibodies), using such solid supports.

### BACKGROUND OF THE INVENTION

Affinity chromatography is composed of a support to which ligands are attached. The ligands specifically bind particular substances which are to be separated and purified (i.e., analytes). Typical examples of a solid supports for affinity chromatography include particles obtained by crosslinking sugar chains (e.g., agarose gel), or particles containing synthetic polymers such as polymethacrylate, polystyrene, polyacyrlamide, silica gels and controlled porous glass. General ligands utilized for affinity chromatography include, but are not limited to, protein A, protein G, glutathione S-transferase, maltose-binding protein, chitin, and immobilized metals, etc.

A support is usually used repeatedly, which typically results in residual amounts of contaminants in the support after a purification operation. Thus, a washing step to remove contaminants can be conducted in effort to restore the support to its original state. Typically, an operation known as cleaning in place (CIP) is carried out with a reagent capable of eluting contaminants from the support. Examples of such reagents include but are not limited to alkaline and acidic liquids, chaotropic agents, urea and guanidine hydrochloride, which effectively remove contaminants such as microorganisms, proteins, lipids and nucleic acids. However, while contaminants are removed from affinity chromatography supports by using alkaline liquids, such alkaline conditions may destabilize a ligand and cause a decrease of its binding capacity.

As antibody based therapies continue to emerge as promising treatments of disease, there is a pressing need for rapid purification of these molecules. Protein A resins for the affinity purification of IgG antibodies have been utilized for the past three decades. However, Protein A resins suffer from relatively low dynamic binding capacities which result in decreased purification efficiency. Further, with the recent advances in cell culture technology, antibody titers have greatly increased, thus placing greater strain on current downstream purification of these molecules. Resins used in such columns are intolerant to the high pH that is necessary for such cleaning, thus limiting the usage lifetime of these resins thereby increasing costs. To mitigate the purification challenges produced by the high demand for antibody-based therapeutics, an affinity chromatography resin that can both efficiently purify large quantities of antibodies and withstand alkaline cleaning procedures would greatly enhance the downstream purification of these molecules.

### SUMMARY OF THE INVENTION

The present invention provides polypeptide affinity ligands. In some embodiments, ligands can be coupled to solid supports for affinity purification of protein analytes, for example, immunoglobulin proteins (e.g., IgG (polyclonal or monoclonal) antibodies). The invention comprises (1) expression and purification of polypeptide affinity ligands, (2) immobilization of polypeptide affinity ligands onto solid support, (3) purification of immunoglobulin proteins and (4) cleaning and reuse of solid support matrices. The polypeptide affinity ligands have increased binding affinity for protein analytes while enabling complete elution/recovery of analytes.

The present invention provides affinity chromatography and methods for isolating protein analytes (e.g., immunoglobulins). The supports for affinity chromatography show excellent alkali resistance, and thereby have high resistance to washing under alkaline conditions. Thus, upon repeated use of the supports for purification of immunoglobulin, the dynamic binding capacity (DBC) for immunoglobulin is not significantly decreased.

### BRIEF DESCRIPTION OF THE FIGURES

FIG. 1 is a graph representing the DBC of a solid support functionalized with polypeptide affinity ligand SEQ ID NO: 3.

FIG. 2 is a graph representing the alkaline stability of a solid support functionalized with polypeptide affinity ligand SEQ ID NO: 3 when treated with 0.1 N sodium hydroxide for 60 minute contact time or with 0.5 N or 1.0 N sodium hydroxide for 15 minute contact time.

FIG. 3 is a graph showing relative DBC for 100 cycles of cleaning with 1.0N NaOH, 1.0N NaOH+1.0M ethylene glycol, 1.0N NaOH+1.0M propylene glycol, 1.0N NaOH+1.0M sucrose.

FIG. 4 is a graph showing host cell protein log reduction value of purification step with varying concentration of arginine in wash buffer. IgG was purified using a pre-packed immobilized ligand in lml column.

### DETAILED DESCRIPTION OF THE INVENTION

In one aspect, the invention relates to polypeptide affinity ligands. In another aspect, the invention relates to methods of purification of polypeptide affinity ligands. In another aspect, the invention relates to the solid support to which the polypeptide affinity ligands are attached. In another aspect, the invention relates to methods of purifying analytes fused to IgG antibodies, using polypeptide affinity ligands attached to solid support matrices. In another aspect, the invention relates to the alkaline stability of the polypeptide affinity ligand when immobilized to the solid support.

Throughout this specification, quantities are defined by ranges, and by lower and upper boundaries of ranges. Each lower boundary can be combined with each upper boundary to define a range. The lower and upper boundaries should each be taken as a separate element.

### Polypeptide Affinity Ligand

A polypeptide affinity ligand of the present invention (herein referred to as “ligand”) specifically binds analytes, e.g., immunoglobulin proteins. The term polypeptide is intended to include any molecule that is comprised of a polypeptide amino acid sequence and structure. The ligands of the invention typically comprise about 80 to about 400 amino acids, and resemble a protein like structure. Examples of other lower boundaries of this range include about 100, about 121, about 150, and about 200 amino acids. Examples of other upper boundaries of this range include about 200, about 300, about 350, and about 391 amino acids. The amino acid sequences of the ligands are arranged in a manner to promote disulfide bond formation that enables enhanced helical structure of a helical domain of a polypeptide. That is, the disulfide bonds stabilize the helical nature of the polypeptide ligand. In some embodiments, typically, there is about one disulfide bond for every about 80-125 amino acids of a ligand. The cysteine amino acids which form a disulfide bond are typically about 28 amino acid residues apart from one another.

In one embodiment, a ligand comprises one to ten of an amino acid sequence herein referred to as “Subunit-1,” and one to ten of an amino acid sequence herein referred to as “Subunit-2.”

The sequences of Subunit-1 have at least about 80% identity, at least about 85% identity, at least about 90% identity, at least about 93% identity, at least about 95% identity, at least about 97% identity, at least about 99% identity, or 100% identity, to SEQ ID NO: 17.

The sequences of Subunit-2 have at least about 80% identity, at least about 85% identity, at least about 90% identity, at least about 93% identity, at least about 95% identity, at least about 97% identity, at least about 99% identity, or 100% identity, to SEQ ID NO: 18, wherein the sequences have two cysteine residues, wherein preferably, the cysteine residues are at positions 5 and 34. Typically, there is a disulfide bond between the two cysteine residues within each Subunit 2 of a ligand.

Subunit 1 and Subunit 2 are arranged in such a manner as to have superior affinity for analytes, while providing stability of a ligand over a wide pH range (3-13).

In one embodiment, the affinity polypeptide ligand comprises one to ten of an amino acid sequence Subunit-1, wherein Subunit-1 has at least about 90% identity to SEQ ID NO: 17, and one to ten of an amino acid sequence Subunit-2, wherein Subunit-2 has at least about 90% identity to SEQ ID NO: 18, wherein residues at positions 5 and 29 are cysteine, wherein there is a disulfide bond between the two cysteine residues, and wherein the ligand comprises at least two of Subunit-1 or at least two of Subunit-2. Preferably, at least one Subunit-1 would separate any two of Subunit-2. That is, a Subunit-1 can follow a Subunit-1 in the sequence of a ligand; however, a Subunit-2 cannot follow a Subunit-2 in the sequence of a ligand.

In some embodiments, Subunit 1 and Subunit 2 are arranged to alternate in sequence in the primary structure of a ligand, and Subunit 1 and Subunit 2 form repeating alpha helical subunits in the secondary structure of a ligand.

In some embodiments, a ligand comprises a spacer between a Subunit-1 and a Subunit-1, and/or between a Subunit-1 and a Subunit-2. A spacer typically comprises about 5 to 20 residues. In some embodiments, a spacer does not comprise cysteine, methionine, proline, aspartic acid or tryptophan. An example of a spacer is SEQ ID NO: 19. Examples of other spacers include a spacer with at least about 80% identity, at least about 85% identity, at least about 90% identity, at least about 93% identity, at least about 95% identity, at least about 97% identity, at least about 99% identity to SEQ ID NO: 19. Different spacers can be used in the same ligand.

In some embodiments, a ligand may further comprise a C-terminal sequence comprised of any natural or unnatural amino acids that serve as a spacer and provide an amino acid anchor residue. For example, the C-terminal amino acid residue can be utilized to immobilize the ligand to a solid support, i.e., the anchor residue can be conjugated to a solid support. In preferred embodiments, the anchor residue is typically an amino acid in which a primary amine is contained within the side chain that can be coupled using techniques known to a skilled artisan, for example, a lysine, serine or alanine. The amino acid spacers are not limited in length. A range of the typical length of a spacer is about 5 to about 25 amino acids. Examples of other lower boundaries of this range include about 7, about 10, and about 15 amino acids. Examples of other upper boundaries of this range include about 16, about 18, and about 20 amino acids.

### Production of Affinity Ligand

In one embodiment, a ligand can be synthetically produced using suitable solid phase peptide synthesis, as would be known by a skilled artisan.

In other embodiments, a ligand can be cloned into a suitable vector and the ligand can be recombinantly expressed in a prokaryotic organism, such as, but not limited to Escherichia coli, Bacillus subtilis, or Staphylococcus aureus. Additionally a eukaryotic expression system, such as fungi (e.g., yeast), insect cells, or mammalian cells may be used for the production of the ligand. For example, known gene recombination technologies that are described in Frederick M. Ausbel, et al., Current Protocols in Molecular Biology, and Sambrook, et al., ed., Molecular Cloning (Cold Spring Harbor Laboratory Press, 3^{rd} edition, 2001) can be used. An expression vector containing a nucleic acid sequence encoding a desired ligand is used for transformation of a host which is then cultured in an appropriate medium to obtain the ligand in large amounts, as is well known in the art.

### Purification of the Affinity Ligand

Following protein expression, a ligand is purified using suitable purification methods to yield pure ligand. The purification strategy is done in such a manner as to promote disulfide bond stabilization of the ligand, e.g., the disulfide bonds within cysteine residues of the ligand, and to retain enhanced affinity of the ligand for analytes, e.g., IgG antibodies. The purification process can involve the following: (1) releasing the intracellular protein from a host cell by disruption using either sonication, chemical lysis, or high pressure homogenization, (2) the addition of highly positively charged polymers ranging in molecular weight from 800 Da to 20,000 Da for the removal of unwanted impurities such as DNA and host cell proteins, (3) clarification of the cell lysate so as to eliminate all other insoluble material from the soluble affinity ligand, (4) purification of the affinity ligand using mixed mode hydrophobic interaction chromatography using or any other equivalent hydrophobic interaction chromatography resin, and (5) final purification of the ligand using high performance anion exchange chromatography.

### Solid Support

Purified ligands of the present invention are attached to a solid support matrix (i.e., “support”). A support can be composed of particles (i.e., porous or non-porous particles). The particles can be in the form of a packed bed or in a suspension form. Suspension forms include a fluidized bed (expanded bed) and a product known as a sheer suspension, and particles can freely be moved about therein. In the case of a packed bed and a fluidized bed, the order of separation steps generally complies with conventional chromatographic methods based on concentration gradients. In the case of the sheer suspension, a batch method is used.

The solid supports can be defined as organic and inorganic supports based on their chemical composition. The solid supports may be based on synthetic polymers or copolymers, including but not limited to polyacrylates, polymethacrylates, polyacrylamide, polymethacrylamide, polyether sulfone, polyethylene, polypropylene, polyamide, polyester, polycarbonate, polyvinyl alcohol, polyvinylether, polystyrene-divinylbenzene copolymer and any derivatives thereof. The solid support may be a natural polymer, including but not limited to polysaccharides (agarose, cellulose, dextran, starch, collagen etc.). The solid support may also be based on inorganic materials, and examples are, but not limited to silica, glass, aluminum oxide, titanium oxide, ceramic, clay and hydroxyapatite. Since almost all affinity separations occur in aqueous solutions, the solid supports generally contains hydrophilic surfaces with various reactive functional groups but not limited to carbonyl (aldehyde or ketone), carboxylic acid, epoxide, hydroxyl, amino groups and their derivatives thereof. These reactive functional groups can facilitate solid supports directly coupling with polypeptide ligands, or after subsequent surface chemical activation under suitable reaction conditions.

The formats of solid supports can be, but not limited to, a bead (spherical or irregular, porous or non-porous), a porous membrane or monolith, a hollow or solid fiber, a chip, slide or plate. The solid supports are often cross-linked with reagents during their preparation process (such as synthetic polymer based matrix) or after formation of matrix (such as polysaccharides based) to provide appreciable porosity and rigidity, benefiting low column operation pressure (e.g., U.S. Pat. No. 4,973,683, incorporated herein by refernce).

A support for affinity chromatography can have a particle size (mean volume diameter) of typically about 20 μm to 200 μm. In the case of preparative scale purification, larger particles are often preferred and a particle size of 30 μm to 100 μm is generally used to achieve high-capacity, large-scale and fast protein purification processes (Handbook of Affinity Chromatography, Toni Kline, p. 24, CRC Press, 1993). The support for affinity chromatography of the present invention is preferably porous with a preferable volume mean pore size of about 50 Å to 4000 Å, as the high surface area of a porous supports enables high protein immobilization amount and binding capacity in their applications. While particles with smaller pores increase their total surface area available for ligand immobilization, they also have higher diffusion resistance for proteins entering the pores. It has previously been reported that the optimum pore size for protein immobilization is generally about 3-5 times the hydrodynamic diameter of the protein ligand. The controlled particle size and pore parameters including pore diameter and distribution of the solid support define the total surface area and flow properties of the support and thus, affect the binding capacity of proteins and downstream purification process efficiency (Anal. Biochem., 406(2):235-237 (2010); J. Chromatogr. A., 888:13-22 (2000); Biotechnol. Equip., 29(2):205-220 (2015)).

### Coupling of Ligand to Solid Support

The solid supports often contain and/or are modified with suitable reactive functional groups for immobilization of the invented polypeptide ligands on the surface. The surface of the supports may be hydrophilic, or modified with hydrophilic reactive functional groups. The activated surface of solid supports may contain various reactive groups but not limited to carbonyl (aldehyde or ketone), epoxide, hydroxyl, carboxylic acid, cyanogen bromide (CNBr), N-hydroxy succinimide ester, carbonyl diimiazole (CDI), organic sulfonate (tosylate and tresylate), azlactone, iodoacetyl, etc. The ligands may be coupled to solid supports by traditional immobilization techniques via single point or multivalent attachment, including but not limited to reductive amination, amine/thiol-epoxide, ‘Click’ chemistry and other substitution reactions. The proper selection of coupling method depends on the characteristics of both solid supports and polypeptide ligands (Affinity Chromatography and Importance in Drug Discovery, Column Chromatography, IntechOpen, DOI: 10.5772/55781).

Coupling can be carried out by using a general method of immobilizing a protein on a support by which the polypeptide ligand is covalently attached and the disulfide bond is not disrupted. For example, in one embodiment, a ligand can be attached using amine and aldehyde chemistry. Coupling can be initiated by first activating a solid support resin with an epoxide functional group. The epoxide functionalization can be achieved by using a linker molecule that is from about 1 to 135 carbons in length, such as a a,w-diglycidyl ether or epichlorohydrin, under basic conditions. The linker molecule sterically promotes the interaction of the ligand with an analyte, e.g., antibodies, during isolation. The epoxide moiety can be converted to a reactive aldehyde group by subsequent ring-opening and oxidation reactions. The amine groups from the side chain of the ligand are then reacted with the aldehyde functionalized solid support in appropriate buffer conditions to form a covalent attachment via a reductive amination reaction. Alternatively, an epoxy-activated solid support as discussed above can be efficiently coupled with amino, thiol or hydroxyl groups of a polypeptide ligand via a one-step nucleophilic substitution reaction under mild coupling conditions. Other examples are using solid supports with carboxyl groups, amino groups or hydroxyl groups after activation by N-hydroxysuccinimide, carbodiimide and cyanogen halide, respectively, followed by coupling with amino or carboxyl groups of a polypeptide ligand via amide bond formation. In addition, hydroxyl group functionalized solid supports after activation by using tosylating or tresylating reagents can also be used to couple with amino or hydroxyl groups of a polypeptide ligand (e.g., U.S. Pat. Nos. 9,040,661 B2; 9,051,375 B2; WO 2011/012302 A1, incorporated herein by reference).

The modification of material surface properties, such as surface hydrophilicity, has been shown to alter the extent of protein adsorption ability (Curr. Top. Med. Chem., 8:270-280 (2008)). The hydroxyl groups produced via a ring-opening reaction of epoxy groups on resin surfaces can increase the hydrophilicity of material, preventing non-specific adsorption of proteins. Therefore, the remaining epoxy groups that are not coupled with polypeptide ligands exist in the support after ligand immobilization and are preferably opened. For example, the remaining epoxy groups can be ring opened by using water as a nucleophilic reagent by stirring the support in water with an acid or an alkali under heating or at room temperature. Alternatively, the remaining epoxy groups may also be blocked by using suitable blocking reagents, such as mercaptoethanol or thioglycerol, and monoethanolamine.

In some circumstances, a linker is often designed and optimized between solid support surface and the affinity ligands, improving orientation flexibility and availability of the attached ligands, thus enhancing the affinity media performance. The linker may comprise any molecule which can be attached to the solid support surface. The linker length effect on affinity media performance was evaluated via activation of resin matrix by different molecules and chemistry. The linker can contain a carbon chain ranging from about 1 to 135 carbons, and may have one or more hetero atoms imbedded in the middle of the linker, such as N, O, S etc. The linker can be a straight or a branched chain with one or multiple reactive groups depending on the chosen solid support activation technique. Examples of linker reagents are but not limited to any α,ω-diglycidyl ethers with various straight carbon chain including polyethyleneglycol, epichlorohydrin, glycidol, α,ω-diamine with various straight carbon chain.

### Methods for Isolating Analytes

Methods for isolating analytes (e.g., IgG antibodies) according to the present invention includes a step of applying a sample containing analytes onto the support to adsorbs the analytes to the support (first step); and a step of eluting the analytes from the support (second step); and further preferably includes a step of washing the support with an alkaline liquid after the second step (third step).

In the first step, a sample containing analytes is applied to, for example, a column filled with the solid support to which the affinity polypeptide has been immobilized for affinity chromatography, under the conditions where the analytes adsorbs to the solid support. In this first step, most of the substances other than the analytes in the sample pass through the column without being adsorbed to the ligand. If necessary, in order to remove some substances that are weakly retained by the ligand, the support may be washed with a neutral buffer solution containing a salt such as sodium chloride.

In the second step, the analytes adsorbed to the solid support are eluted by applying appropriate buffer solutions at about pH 3 to 5. By collecting the eluent, analytes can be isolated from the sample.

In the third step, the support is washed with an alkaline liquid (CIP washing). Examples of the alkaline liquid used in the third step include an aqueous sodium hydroxide solution, an aqueous potassium hydroxide solution, triethylamine, and tetrabutylammonium hydroxide.

The support for affinity chromatography of the present invention stably retains the analytes binding ability due to the high alkali resistance of the ligands even after the washing in the third step; therefore, the support can be repeatedly used for analyte isolation methods of the present invention.

In some embodiments, the affinity chromatography solid support according to the present invention has high DBC for IgGs with a capacity of greater than about 30 g/L and shows high resistance against washing under alkaline conditions (e.g., washing using an alkaline liquid such as sodium hydroxide solution at 0.01N to 1.0N) which provides ≥100 cycles of cleaning with 0.5N sodium hydroxide with 15 minute contact time while retaining 90% effectiveness for binding analytes.

### Additives to Improve Purification Performance

Common analytes that are purified using the solid supports in the present invention are IgG antibodies, which require removal of endotoxin, host cell proteins, DNA fragments, cell culture media components and other product related impurities such as aggregates or degradation products. After loading unpurified antibody onto the support, a wash step is utilized to clear out impurities. Commonly known wash buffers include PBS buffer, tris buffers, HEPES buffer in the pH range of 4.5 to 9.0 with or without salts such as sodium chloride, sodium sulfate, potassium chloride.

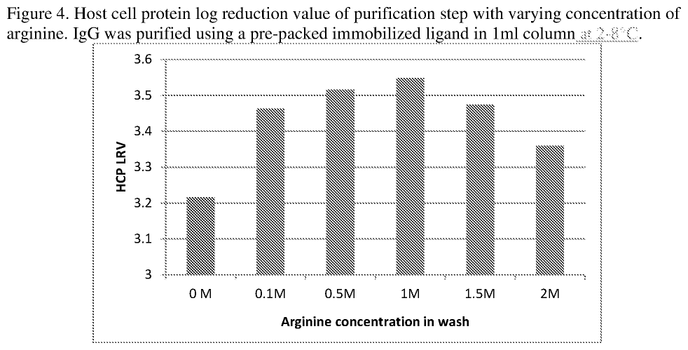

Certain additives used in the antibody purification process utilizing the affinity resin prepared by immobilizing affinity ligands as disclosed in this application surprisingly significantly improve impurity clearance and increased purity of antibody without significantly impacting yield. These additives when added to wash buffer having pH in the range of about 4.5 to 10.0 remove impurities that are either strongly interact with affinity ligand or solid support. The additives shown to be effective, individually or in-combination, are illustrated in FIG. 4 and Table 1.

In one embodiment of the instant invention, additive compositions (i.e., “additives”) are provided for use in the wash step to improve the impurity clearance. Additives in the wash buffer can be used individually or as a mixture. Additives include amino acids such as arginine, acetyl arginine or propylene glycol, tween 20, guanidine HCl, urea, or IPA.

In some embodiments, the additive solution includes about 1% to 10% of propylene glycol; 0.1M to 1M Arginine; 0.1% to 1% of tween 20; 0.1M to 1M of guanidine HC1; 1% to 10% of IPA; 1.0M to 3.0M of urea.

### Additives to Improve Stability of Immobilized Affinity Ligand during Alkaline Treatment

Solid supports with immobilized affinity ligands are usually used repeatedly for purifying analytes, which typically results in residual amounts of contaminants in the supports. A cleaning step (alkaline treatment step) is used at the end to remove such contaminants; however, cleaning solutions are typically highly alkaline which affects the stability of solid supports/affinity ligands.

In one embodiment of the instant invention, additive compositions (i.e., “additives”) are provided for use in the cleaning step to maintain the stability of supports/ligands while effectively removing contaminants. That is, the additives protect the immobilized affinity ligands and inhibit their degradation.

The additives can be included in high alkaline solutions, such as, e.g., one molar sodium hydroxide or higher. For example, when the concentration of NaOH is in the range of about 0.1N to 1.0N, the concentration of additives can be in the range of about 0.1M to 2.0M. The stabilizing effect can be modulated by utilizing specific concentration of the additive depending on the concentration of alkaline solutions. For example, 1M solution of sucrose additive is effective for a 1N NaOH solution.

The additives comprise carbohydrates (e.g., sucrose, trehalose), polyols, glycols (e.g., ethylene glycol, propylene glycol) and/or amino acids (e.g., arginine, acetyl arginine).

In one embodiment, the additive solution includes about 1.0M Ethylene glycol with 1.0N NaOH; 1.0M Propylene glycol with 1.0N NaOH; 1.0M Sucrose with 1.0N NaOH.

### Percent Identity

The sequences falling within the scope of the present invention are not limited to the specific enumerated sequences, but also include variations thereof. Preferred computer program methods to determine identity between two sequences include, but are not limited to, BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NILM/NIH Bethesda, Md. 20894; Altschul et al., supra). The well known Smith Waterman algorithm may also be used to determine identity.

### EXAMPLES

### Example 1

### Molecular Cloning and Expression of Affinity Ligand

### Materials and Methods

Genes encoding for the affinity ligands were codon optimized for recombinant expression in E. coli. These genes were synthesized are then cloned into pET24a (Novagen) expression vector using Ndel and Xhol restriction sites (Synbio). Following ligation, the expression vector is transformed into BL21 DE3 competent E. coli by heat shocking the bacteria at 42° C. for 45 seconds. The transformed cultures are incubated in LB medium for 1 hour at 37° C. and 250 rpm. After incubation, the bacteria is streaked onto LB agar with 50 μg/mL kanamycin plates (Teknova) and allowed to incubate at 37° C. overnight. Individual colonies are then chosen for expression verification.

A single colony of E. coli transformed with the affinity ligand of interest is selected from a 50 m/mL kanamycin plate (Teknova) that was grown overnight. This colony is used to inoculate a fresh culture of LB media (Sigma) and is then incubated at 37° C. with shaking until the culture has grown to an optical density of −0.6. At this point, 1 mM isopropyl-beta-D-thiogalactopyranoside (IPTG) (Avantor) is introduced to the culture. Following the addition of IPTG, the culture is incubated at 37° C. with shaking for an additional 3 hours. The cells are then harvested by centrifugation at 6000×g for 15minutes and expression is confirmed using SDS-PAGE.

Variants that were expressed were then characterized for expression levels through use of an IgG affinity chromatography column (GE). A solution of 50 mM Tris, 50 mM Sodium Chloride, pH 7.4 is added to a ˜2-3 gram sample of harvested cells until 10% solids is reached. Mixture is homogenized using a turrax rotary homogenizer. The homogenized solution is subsequently passed through a cell homogenizer (Microfluidics Model 110Y homogenizer) three times at 10,000PSI to lyse the cells. Lysed cell solution is clarified by centrifugation at 20,000×g at 4° C. for 30 minutes. Supernatant material is poured into a tared, sterile container and the net mass is recorded. Using the mass of initial cells added to create 10% solution, the supernatant concentration (supernatant/g of cell paste) is calculated. The amount of supernatant loaded onto the IgG Affinity column is calculated such that it is the equivalent to the supernatant obtained from 0.5 g of cell paste. The chromatography process utilizes 1× Phosphate Buffered Saline pH 7.4 (PBS) and 100 mM Acetic Acid pH 3.4 (HAc) as load and elution buffers, respectively. The column is stripped of any bound material with 3 Column Volumes (CV) of HAc and equilibrated with 10 CV of PBS. The prior calculated amount of supernatant sample is loaded directly onto the column and then the column is washed with 5 CV of PBS. The sample is then eluted using 5 CV of HAc. Subsequently, the column is equilibrated with 5 CV of PBS. The area of the elution UV peak is calculated using automatic fraction integration, and the amount of target ligand is calculated using the polypeptide extinction coefficient. This amount of target protein is then normalized to give the final target polypeptide ligand expression as mg of target ligand polypeptide ligand/g of cell paste.

### Example 2

### Purification of Affinity Ligand

Cells expressing soluble affinity ligand of interest are resuspended in buffer and lysed utilizing chemical, enzymatic, or mechanical means which can include pressure, shear force, or sonication. The resulting cell lysate is prepared for chromatography through various means such as clarification through centrifugation, depth filtration, PEI precipitation of DNA, or fractionation of proteins through ammonium sulfate or PEG. The protein target is then purified through chromatographic techniques such as IMAC affinity, size exclusion, ion exchange, hydrophobic interaction, or a combination of the above. Buffers can be exchanged between steps through use of UFDF/TFF, dilution, or dialysis to change the pH and/or ionic strength, or remove additives.

More specifically, frozen cell paste is suspended in a 50mM Tris, 50 mM NaCl, pH 8.0 solution to yield a 10-20% solids suspension solution. The cells are passed 2-5 times through a homogenizer (Microfluidics Model 110Y homogenizer) at 10,000 psi to generate a cell lysate solution. 0.25% PEI (w/v) is added to the solution and mixed for 30 minutes. The solution is subsequently centrifuged at 15,000×g for 15-30 minutes at 4° C. to yield a clarified cell lysate solution. 0.08g of ammonium sulfate/mL of clarified cell lysate solution is added and mixed for 30 minutes prior to sterile filtration. The resulting solution is subsequently incubated at room temperature for 6 -24 hours.

Following incubation, the clarified cell lysate material is loaded onto a packed BAKERBOND™ polyHiPropyl column equilibrated with 25 mM tris, 0.6 M ammonium sulfate, pH 8.0 at a concentration of 1.0 g of cell paste per ml of resin. The column is then washed with 10 CV of 25 mM tris, 0.6M ammonium sulfate, pH 8.0. The column is subsequently treated to elute the target polypeptide ligand using a gradient of 100% 25 mM tris, 0.6M ammonium sulfate, pH 8.0 to 100% 25 mM tris, pH 8.0 over 1 CV followed by an additional 4 CV of 100% 25 mM tris, pH 8.0. The material is then buffer exchanged to a final concentration of 5-10 mg total protein/mL using a 25 mM tris, pH 9.0 solution and a 3-5 kDa molecular weight cut off (MWCO) ultrafiltration/diafiltration (UFDF) membrane. The buffer exchanged material is loaded onto a packed BAKERBOND™ PolyQuat column and equilibrated with 25 mM tris, pH 9.0 at a concentration up to 20 mg/ml of resin. The column is subsequently washed with 5 CV 25 mM tris, 25 mM sodium acetate, pH 9.0. The loaded target protein is then eluted from the column via a gradient elution of 25 mM-137 mM sodium acetate, pH 9.0 over 3 CV followed by a 10 CV gradient to 150 mM sodium acetate, pH 9.0. The target polypeptide ligand is then buffer exchanged into 1×PBS via UFDF using a 3-5 MWCO membrane. Following purification, the affinity ligand is characterized by mass spectrometry to confirm disulfide bond formation.

### Example 3

### Immobilization of Ligand to Aldehyde Functionalized Solid Support

Agarose resin particles (20 mL, 50-85 μm), epichlorohydrin (4.45-23.6 g), and aqueous NaOH solution (20 mL, 0.6-2.5 M) were mixed in a 50-mL centrifuge tube and placed on a mechanical shaker at 200 rpm for 4 hours at 35° C. The mixture was removed from shaker, filtered in a fritted glass funnel (medium porosity) and washed with DI water (40 mL×3), 50% alcohol/water (40 mL×1), alcohol (40 mL×2) and DI water (40 mL×3). Then the resin was washed with aqueous H₂SO₄ (pH=2.5) and transferred back into a 100-mL tube by rinsing with aqueous H₂SO₄ solution. The mixture (˜50 mL) was placed in the shaker (60° C.) and shaken at ˜225 rpm overnight. The mixture was filtered, washed with DI water (20 mL×6) and transferred into a 100-mL tube. The resin was sedimented and decanted carefully before adding sodium periodate solution (60 mL, 0.4 M), and the mixture was shaken for 5 hours at room temperature. The resin was filtered, washed with DI water and stored in DI water in a refrigerator for future use.

A slurry of aldehyde functionalized agarose (1.5 mL resin) was taken in a 15 ml centrifuge tube. It was centrifuged at 500 rpm for 5 min before supernatant was removed. 3 ml of 2 M Guanidine Hydrochloride in 0.1 M Na₂HPO₄ (pH=6.7) was added to the resin, mixed and centrifuged followed by the removal of supernatant. The slurry was buffer exchanged two more times with same salt solution. The supernatant was removed and ligand (15-22.5 mg.) was added to the resin. The mixture was shaken in a vertical shaker for 3 h followed by the addition of NaBH₃CN in 0.1 M Na₂HPO₄ (2mL, 100 mg/mL, pH=7) and shaking was continued for 1 h at room temperature. This slurry was quenched by shaking for 1 h after adding 0.9 ml of 1% NH₂EtOH in water. The slurry was then centrifuged and supernatant collected to calculate amount of unbound polypeptide by HPLC. The slurry was then washed three times with 0.1 M Na₂HPO₄ (pH=7), 0.2 M Acetate (pH=3) and 1×PBS (pH=7.2). The final resin was kept as 50% solution in 1×PBS and stored at 4° C.

### Example 4

### Immobilization of Ligand to Epoxy Functionalized Solid Support

Agarose resin particles (20 mL, 50-85 μm), ethylene glycol diglycidyl ether (2.8-8.4 g), and aqueous NaOH solution (20 mL, 0.6 M) were mixed in a 50-mL centrifuge tube and placed on a mechanical shaker at 200 rpm for 4-8 hours at room temperature. The mixture was removed from shaker, filtered in a fritted glass funnel (medium porosity) and washed with deionized (DI) water (20 mL×5), 50% isopropyl alcohol/water (50 mL×1), isopropyl alcohol (50 mL×3). The resulting resin was stored in isopropyl alcohol in a refrigerator for future use.

A slurry of epoxide activated agarose (1.5 mL resin) in isopropyl alcohol was centrifuged in a 15-mL centrifuge tube at 600 rpm for 5 mins. The supernatant was removed carefully and 3.0-4.5 mL of 1.2M of Na₂SO₄ in 0.15M Na₂CO₃ (pH 11) was added and mixed with the resin. The resulting slurry was centrifuged at 500 rpm for 5 min before second buffer exchange. This buffer exchange process was repeated three times and the final resin volume of agarose resin and buffer was adjusted to ˜1.8 mL carefully. Polypeptide ligand (15-22.5 mg) was added followed by the addition of 170-256 mg of Na₂SO₄ and 24-36 mg of Na₂CO₃ and the resulting mixture was shaken for 7-24 h at room temperature. The slurry was centrifuged to collect supernatant for calculating the amount of bound ligand by HPLC. The slurry was washed three times with 4.5 mL DI H₂O via centrifuging. Slurry was subsequently buffer exchanged by using 4.5 mL of 1×PBS (pH=7.2) three times via centrifuging. The final resin was kept as 50% solution in 1×PBS and stored at 4° C.

### Example 5

### Dynamic Binding Capacity

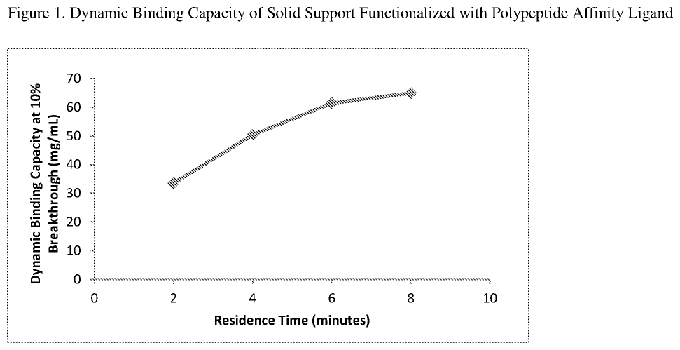

The DBC of the solid support functionalized with affinity polypeptide ligand to bind IgG was evaluated at 2-8° C. (FIG. 1). 1 ml of solid support resin with immobilized ligand is packed into 1 ml columns and equilibrated in 1×PBS. 2 mg/ml IgG prepared in 10 mM Na₂HPO₄, 2 mM NaH₂PO₄, 137 mM NaCl pH 7.4 buffer is loaded onto the column either at 0.125 ml/min, 0.167 ml/min, 0.25 ml/min or 0.5 ml/min until 10% of the IgG A280 max is observed. The target IgG protein is eluted with 100 mM acetic acid for 10 CV and re-equilibrated in 10 CV 1×PBS. The DBC of the solid support is determined by first subtracting the system void volume from the IgG load volume at 10% breakthrough and then multiplying by the concentration of the IgG load solution.

### Example 6

### Alkaline Stability Analysis

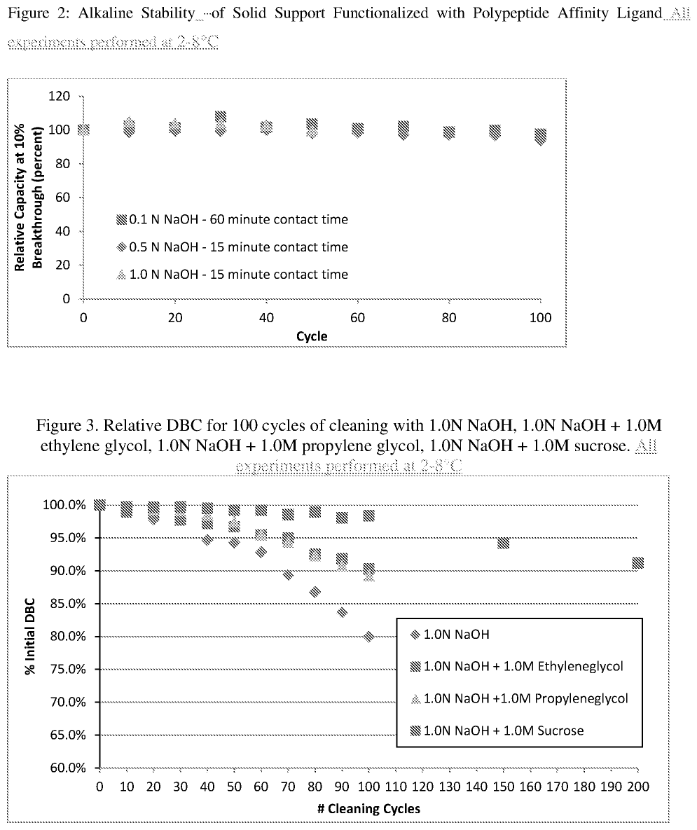

Alkaline stability of the solid support with immobilized ligand was determined at 2-8° C. using 0.1N, 0.5N and 1.0N NaOH (FIG. 2). In these experiments, the solid support with immobilized ligand is first equilibrated with 1×PBS, loaded with IgG, washed with 1×PBS, and treated with acetic acid to elute the IgG as stated in Example 5. Following elution of the IgG with acetic acid, the column is equilibrated with 1×PBS and subsequently treated with either 0.1N, 0.5N or 1.0N NaOH. When using 0.5N or 1.0N NaOH, a flow rate sufficient to achieve a 15 minute contact time is utilized to complete one cycle of alkaline treatment. When using 0.1N NaOH, 10 CV is passed over the resin bed and the flow rate is then paused to ensure 60 minutes contact time to complete one cycle of alkaline treatment. The process of treating the column with 1X PBS and sodium hydroxide is repeated until a total of ten alkaline treatments of the solid support are completed. Following the ten alkaline treatment cycles of the column, the solid support is then equilibrated with 1×PBS, loaded with IgG, washed with 1×PBS, treated with acetic acid, and followed by an additional 10 PBS/NaOH treatments of the column as stated above. This process is repeated for a total of 100 alkaline treatment cycles with 0.1N NaOH and 0.5N NaOH and for a total of 50 alkaline treatment cycles when using 1.0 N NaOH. The DBC of the solid support is determined as stated in Example 5 after 0, 10, 20, 30, 40, 50, 60, 70, 80, 90 and 100 alkaline treatment cycles. Alkaline stability of the solid support is determined through the loss of DBC following treatment with NaOH.

### Example 7

### Additive to Increase Stability during Alkaline Treatment

This example illustrates the effect of additive on increasing the stability of solid support prepared by immobilizing peptide affinity ligand.

FIG. 3 shows % DBC (dynamic binding capacity) when additives of 1.0M Ethylene glycol or 1.0M Propylene glycol or 1.0M Sucrose was used with 1.0 N NaOH cleaning solution at 2-8° C. Each cleaning cycle was performed by flowing cleaning solution mixture though lml of pre-packed column with immobilized ligand with 15 min of contact time. DBC was measured at 10% of breakthrough at every 10 cleaning cycles.

When the column was cleaned with only 1.0N NaOH, DBC decreased to 80% of initial value after 100cycles of cleaning. When the column was cleaned with 1.0N NaOH with 1.0M Ethylene glycol, DBC decreased to 90.3% of initial value after 100 cycles of cleaning. When the column was cleaned with 1.0N NaOH with 1.0M propylene glycol, DBC decreased to 89.2% of initial value after 100 cycles of cleaning. Lastly, when the column was cleaned with 1.0N NaOH with 1.0M sucrose, DBC decreased to 98.4% of initial value after 100 cycles of cleaning. The data show that when additives are used in the cleaning step with 1.0N NaOH, the stability of resin is increased and remaining dynamic binding capacity is higher than cleaning with 1.0N NaOH alone.

### Example 8

### Addition of Arginine at Wash Step to Improve HCP Removal

Removal of host cell protein (HCP) was analyzed at 2-8° C. while arginine concentration was varied in wash step. Purification was performed by packing lml of immobilized ligand and equilibrating with 1×PBS, then the column was loaded with unpurified antibody (feedstock) and washed with 1×PBS or 1×PBS mixture with arginine. Elution was performed by flowing acetic acid solution through the column as stated in Example 5. The elution pool was analyzed for HCP concentration. FIG. 4 shows the effect of addition of arginine to 1'PBS wash buffer to maximize the HCP log reduction value (LRV). When wash step was performed with 1X×PBS buffer, HCP LRV was 3.22, while addition of arginine increased the LRV regardless of concentration. Most significant effect was observed when concentration of arginine was at 1M, where LRV increased to 3.55.

### Example 9

### Addition of Mixture of Additives to Improve HCP Removal

Removal of host cell protein (HCP) was analyzed at 2-8° C. while component, pH and concentration were varied in the wash step. Purification was performed by packing lml of immobilized ligand column and equilibrating with 1×PBS. The column was loaded with IgG feedstock and washed with 1×PBS followed by buffer with additives and final wash with 1×PBS. Elution was performed by flowing acetic acid buffer through the column as stated in Example 5. The elution pool was analyzed for HCP concentration. When 1×PBS was used for wash step as a control, HCP LRV (log reduction value) was at 3.49. It was surprisingly observed that the wash step with different concentrations of propylene glycol, tween 20, guanidine HCl, arginine, IPA, urea and isopropanol as additives increases LRV without impacting yield of monoclonal antibody, (i.e., there is no significant elution of antibody from the resin and yield was maintained above 95%). List of wash additives tested with resulting LRV are shows in Table 1. The most significant increase in LRV was observed with additive mixture of 10% isopropanol, 3M Urea at pH7.4, where LRV was increased to 3.82.

TABLE 1
Host cell protein log reduction value of purification step
with varying wash buffer components. IgG was purified using
a pre-packed immobilized ligand in 1 ml column at 2-8° C.

| Wash buffer | HCP LRV |
|---|---|
| Control (1x PBS, pH 7.4) | 3.49 |
| 1x PBS, 10% propylene glycol, 1M Arginine, | 3.65 |
| 1% tween 20, pH 7.4 | |
| 1x PBS, 1M Guanidine HCl, pH 7.4 | 3.72 |
| 1x PBS 10% propylene glycol, 1M Guanidine | 3.76 |
| HCl, pH 7.4 | |
| 1x PBS, 10% propylene glycol, 1M Arginine, | 3.66 |
| pH 7.4 | |
| 1X PBS, 0.5M Arginine, pH 10 | 3.67 |
| 1X PBS, 1M Arginine, pH 7.4 | 3.65 |
| 1X PBS, 10% isopropanol, 1M Arginine, | 3.81 |
| pH 7.4 | |
| 1X PBS, 10% isopropanol, 1M Arginine, 1% | 3.73 |
| Tween20, pH 7.4 | |
| 1X PBS, 1M Urea, pH 7.4 | 3.61 |
| 1X PBS, 10% isopropanol, 1M urea, pH 7.4 | 3.67 |
| 1X PBS, 10% isopropanol, 2M urea, pH 7.4 | 3.77 |
| 1X PBS, 10% isopropanol, 2M urea, 1% Tween20, | 3.75 |
| pH 7.4 | |
| 1X PBS, 10% isopropanol, 3M urea, pH 7.4 | 3.82 |
| 1X PBS, 10% isopropanol, 3M urea, 1% tween20, | 3.81 |
| pH 7.4 | |

### INCORPORATION OF SEQUENCE LISTING

Incorporated herein by reference in its entirety is the Sequence Listing for the application. The Sequence Listing is disclosed on a computer-readable ASCII text file titled, “Sequence_Listing_2198-205PCT.txt”, created on Feb. 4, 2021. The sequence_listing.txt file is 37.1 KB in size.

### Polypeptide Affinity Ligands Sequence Listing

Sequence: 1
Length: 121
Organism: Artificial Sequence
(SEQ ID NO: 1)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSK
Sequence: 2
Length: 160
Organism: Artificial Sequence
(SEQ ID NO: 2)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSK
Sequence: 3
Length: 198
Organism: Artificial Sequence
(SEQ ID NO: 3)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSK
Sequence: 4
Length: 237
Organism: Artificial Sequence
(SEQ ID NO: 4)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSFNMQCQRRFYEALHDPNLTEEQRNAKIQSIRDDCAVAQSK
Sequence: 5
Length: 275
Organism: Artificial Sequence
(SEQ ID NO: 5)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSFNMQCQRRFYEALHDPNLTEEQRNAKIQSIRDDCAVAQSFNMQQQRRFYEALHDPNLT
EEQRNAKIQSIRDDAVAQSK
Sequence: 6
Length: 314
Organism: Artificial Sequence
(SEQ ID NO: 6)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSK
Sequence: 7
Length: 352
Organism: Artificial Sequence
(SEQ ID NO: 7)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSK
Sequence: 8
Length: 391
Organism: Artificial Sequence
(SEQ ID NO: 8)
AVAQSFNMQQQRRFYEALHDPNLTEEQRNAKIQSIRDDAVAQSFNMQCQRRFYEALHDPNLTE
EQRNAKIQSIRDDCAVAQSFNMQQQRRFYEALHDPNLTEEQRNAKIQSIRDDAVAQSFNMQCQR
RFYEALHDPNLTEEQRNAKIQSIRDDCAVAQSFNMQQQRRFYEALHDPNLTEEQRNAKIQSIRDD
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSK
Sequence: 9
Length: 122
Organism: Artificial Sequence
(SEQ ID NO: 9)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSK
Sequence: 10
Length: 160
Organism: Artificial Sequence
(SEQ ID NO: 10)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSK
Sequence: 11
Length: 199
Organism: Artificial Sequence
(SEQ ID NO: 11)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSK
Sequence: 12
Length: 237
Organism: Artificial Sequence
(SEQ ID NO: 12)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSFNMQQQRRFYEALHDPNLTEEQRNAKIQSIRDDAVAQSK
Sequence: 13
Length: 276
Organism: Artificial Sequence
(SEQ ID NO: 13)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSFNMQQQRRFYEALHDPNLTEEQRNAKIQSIRDDAVAQSFNMQCQRRFYEALHDPNLT
EEQRNAKIQSIRDDCAVAQSK
Sequence: 14
Length: 314
Organism: Artificial Sequence
(SEQ ID NO: 14)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSFNMQQQRRFYEALHDPNLTEEQRNAKIQSIRDDAVAQSFNMQCQRRFYEALHDPNLT
EEQRNAKIQSIRDDCAVAQSFNMQQQRRFYEALHDPNLTEEQRNAKIQSIRDDAVAQSK
Sequence: 15
Length: 353
Organism: Artificial Sequence
(SEQ ID NO: 15)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSFNMQQQRRFYEALHDPNLTEEQRNAKIQSIRDDAVAQSFNMQCQRRFYEALHDPNLT
EEQRNAKIQSIRDDCAVAQSFNMQQQRRFYEALHDPNLTEEQRNAKIQSIRDDAVAQSFNMQCQ
RRFYEALHDPNLTEEQRNAKIQSIRDDCAVAQSK
Sequence: 16
Length: 391
Organism: Artificial Sequence
(SEQ ID NO: 16)
AVAQSFNMQCQRRFYEALHDPNLTEEQRNAKIQSIRDDCAVAQSFNMQQQRRFYEALHDPNLT
EEQRNAKIQSIRDDAVAQSFNMQCQRRFYEALHDPNLTEEQRNAKIQSIRDDCAVAQSFNMQQQ
RRFYEALHDPNLTEEQRNAKIQSIRDDAVAQSFNMQCQRRFYEALHDPNLTEEQRNAKIQSIRDD
CAVAQSFNMQQQRRFYEALHDPNLTEEQRNAKIQSIRDDAVAQSFNMQCQRRFYEALHDPNLT
EEQRNAKIQSIRDDCAVAQSFNMQQQRRFYEALHDPNLTEEQRNAKIQSIRDDAVAQSFNMQCQ
RRFYEALHDPNLTEEQRNAKIQSIRDDCAVAQSFNMQQQRRFYEALHDPNLTEEQRNAKIQSIRD
DAVAQSK
Sequence: 17
Length: 33
Organism: Artificial Sequence
(SEQ ID NO: 17)
FNMQQQRRFYEALHDPNLTEEQRNAKIQSIRDD
Sequence: 18
Length: 34
Organism: Artificial Sequence
(SEQ ID NO: 18)
FNMQCQRRFYEALHDPNLTEEQRNAKIQSIRDDC
Sequence: 19
Length: 5
Organism: Artificial Sequence
(SEQ ID NO: 19)
AVAQS.

1. An affinity polypeptide ligand comprising:
one to ten of Subunit-1, wherein Subunit-1 has at least about 90% identity to SEQ ID NO:
17; and one to ten of Subunit-2, wherein Subunit-2 has at least about 90% identity to SEQ ID NO: 18, wherein residues at positions 5 and 34 are cysteine, wherein there is a disulfide bond between the two cysteine residues; and
wherein the ligand comprises at least two of Subunit-1 or at least two of Subunit-2, wherein at least one Subunit-1 would be between any two of Subunit-2.

2. The affinity polypeptide ligand of claim 1 comprising an amino acid spacer between Subunit-1 and Subunit-1, and between Subunit-1 and Subunit-2, wherein the spacer comprises about 5 to 20 residues.

3. An affinity polypeptide ligand according to claim 2 wherein a C-terminal amino acid residue immobilizes the ligand to a solid support.

4. An affinity polypeptide ligand according to claim 1 having at least about 90% identity to SEQ ID NO: 3, wherein a disulfide bond is formed between the cysteine residues at positions 48 and 77, and at positions 125 and 154.

5. An affinity polypeptide ligand according to claim 1 having at least about 90% identity to SEQ ID NO: 1, wherein a disulfide bond is formed between the cysteine residues at positions 48 and 77.

6. An affinity polypeptide ligand according to claim 1 having at least about 90% identity to SEQ ID NO: 2, wherein a disulfide bond is formed between the cysteine residues at positions 48 and 77, and at positions 125 and 154.

7. An affinity polypeptide ligand according to claim 1 having at least about 90% identity to SEQ ID NO: 4, wherein a disulfide bond is formed between the cysteine residues at positions 48 and 77, at positions 125 and 154, and at positions 202 and 231.

8. An affinity polypeptide ligand according to claim 1 having at least about 90% identity to SEQ ID NO: 5, wherein a disulfide bond is formed between the cysteine residues at positions 48 and 77, at positions 125 and 154, and at positions 202 and 231.

9. An affinity polypeptide ligand according to claim 1 having at least about 90% identity to SEQ ID NO: 6, wherein a disulfide bond is formed between the cysteine residues at positions 48 and 77, at positions 125 and 154, at positions 202 and 231, and at positions 279 and 308.

10. An affinity polypeptide ligand according to claim 1 having at least about 90% identity to SEQ ID NO: 7, wherein a disulfide bond is formed between the cysteine residues at positions 48 and 77, at positions 125 and 154, at positions 202 and 231, and at positions 279 and 308.

11. An affinity polypeptide ligand according to claim 1 having at least about 90% identity to SEQ ID NO: 8, wherein a disulfide bond is formed between the cysteine residues at positions 48 and 77, at positions 125 and 154, at positions 202 and 231, at positions 279 and 308, and at positions 356 and 385.

12. An affinity polypeptide ligand according to claim 1 having at least about 90% identity to SEQ ID NO: 9, wherein a disulfide bond is formed between the cysteine residues at positions 10 and 39, and at positions 87 and 116.

13. An affinity polypeptide ligand according to claim 1 having at least about 90% identity to SEQ ID NO: 10, wherein a disulfide bond is formed between the cysteine residues at positions 10 and 39, and at positions 87 and 116.

14. An affinity polypeptide ligand according to claim 1 having at least about 90% identity to SEQ ID NO: 11, wherein a disulfide bond is formed between the cysteine residues at positions 10 and 39, at positions 87 and 116, and at positions 164 and 193.

15. An affinity polypeptide ligand according to claim 1 having at least about 90% identity to SEQ ID NO: 12, wherein a disulfide bond is formed between the cysteine residues at positions 10 and 39, at positions 87 and 116, and at positions 164 and 193.

16. An affinity polypeptide ligand according to claim 1 having at least about 90% identity to SEQ ID NO: 13, wherein a disulfide bond is formed between the cysteine residues at positions 10 and 39, at positions 87 and 116, at positions 164 and 193, and at positions 241 and 270.

17. An affinity polypeptide ligand according to claim 1 having at least about 90% identity to SEQ ID NO: 14, wherein a disulfide bond is formed between the cysteine residues at positions 10 and 39, at positions 87 and 116, at positions 164 and 193, and at positions 241 and 270.

18. An affinity polypeptide ligand according to claim 1 having at least about 90% identity to SEQ ID NO: 15, wherein a disulfide bond is formed between the cysteine residues at positions 10 and 39, at positions 87 and 116, at positions 164 and 193, at positions 241 and 270, and at positions 318 and 347.

19. An affinity polypeptide ligand according to claim 1 having at least about 90% identity to SEQ ID NO: 16, wherein a disulfide bond is formed between the cysteine residues at positions 10 and 39, at positions 87 and 116, at positions 164 and 193, at positions 241 and 270, and at positions 318 and 347.

20. A method for isolating a polypeptide ligand of claim 1, wherein the polypeptide ligand is purified using cationic additives, hydrophobic interaction chromatography, and/or ion exchange chromatography.

21. A solid support that is porous in nature to which the affinity polypeptide ligand of claim 1 is immobilized.

22. The solid support of claim 21 wherein the solid support is functionalized with an aldehyde moiety to which the affinity polypeptide ligand is attached.

23. The solid support of claim 21 wherein the solid support is functionalized with an epoxy moiety to which the affinity polypeptide ligand is attached.

24. The solid support of claim 21, wherein the ligand has at least 90% identity to SEQ ID NO: 3, wherein a disulfide bond is formed between the cysteine residues at positions 48 and 77, and at positions 125 and 154.

25. The solid support of claim 21, wherein the ligand has at least 90% identity to SEQ ID NOs: 1, 2, 4, 5, 6, 7, 8, 9, 10, 11, 12, 13, 14, 15 or 16, wherein disulfide bonds stabilize the helical nature of the polypeptide and wherein the ligand is bound to the solid support.

26. The solid support of claim 21 wherein the support binds greater than about 30 g/L of immunoglobulin protein analyte.

27. The solid support of claim 21 wherein the support withstands 0.1M-1.0M sodium hydroxide alkaline cleaning solutions.

28. The solid support of claim 21 wherein the support removes host cell impurities, including DNA, endotoxin, cell culture media components and other non-monoclonal antibody proteins.

29. A method for isolating an analyte comprising: applying a sample containing the analyte through the solid support of claim 21 to adsorb the analyte to the support; and eluting the adsorbed analyte from the support.

30. The method according to claim 29 further comprising washing impurities with a buffer before eluting the adsorbed analyte from the support.

31. The method according to claim 29, wherein the analyte is an immunoglobulin.

32. The method according to claim 30 wherein the buffer contains additives.

33. The method according to claim 32, wherein the additives comprise about 1% to 10% propylene glycol; about 0.1M to 1M Arginine; about 0.1% to 1% of tween 20; about 0.1M to 1M of guanidine HC1; about 1% to 10% of IPA and/or about 1M to 3M of urea.

34. A method of cleaning the solid support of claim 21 where the support is contacted with a cleaning solution comprising about 1 M-2 M sodium hydroxide alkaline.

34. ethod of claim 34 wherein the cleaning solution comprises a carbohydrate, a polyol, a glycol and an amino acid.

36. The method of claim 35 wherein the carbohydrate is sucrose and/or trehalose.

37. The method of E2 wherein the glycol is ethylene glycol and/or propylene glycol.

38. The method of claim 35 wherein the amino acid is arginine and/or acetyl arginine.

39. The method of claim 34 the cleaning solution comprises about 1.0M Ethylene glycol with 1.0N NaOH; about 1.0M Propylene glycol with 1.0N NaOH; and about 1.0M Sucrose with 1.0N NaOH.
